(19) United States
(12) Patent Application Publication
(10) Pub. No.: US 2022/0208324 A1
(43) Pub. Date: Jun. 30, 2022

(54) EMR INTEGRATED ELECTRONIC CASE REPORTING

(71) Applicant: CERNER INNOVATION, INC., KANSAS CITY, KS (US)

(72) Inventors: Rajneesh Mehra, Overland Park, KS (US); Todd Wyeth Fritsche, Phoenixville, PA (US); Steven Ger, Philadelphia, PA (US); Nigel Meachen, Phoenixville, PA (US); Jesse Wyatt, Kansas City, MO (US); Tod Ryal, Liberty, MO (US); Elizabeth Hough, Montgomery, PA (US); Jerald Greg Mahurin, Lee's Summit, MO (US); Kirsten Hagemann, Shawnee, KS (US); Matthew Birkel, Mission, KS (US); Susan McKernan, Kansas City, KS (US); Girish Chawla, Collegeville, PA (US); Arvindo Kinny, Malvern, PA (US); Sweta Dwivedi, Overland Park, KS (US)

(21) Appl. No.: 17/137,721

(22) Filed: Dec. 30, 2020

(51) Int. Cl. G16H 15/00; H04L 29/06; G06F 9/54; G16H 10/60; G16H 10/40; G16H 50/70; G16H 70/20; G16H 70/60; G16H 40/20; G06Q 50/26; G06Q 10/10; G16H 50/80; G16H 70/40

(52) U.S. Cl. CPC G16H 15/00; H04L 63/166; G06F 9/541; G16H 10/60; G16H 10/40; G16H 50/70; A61B 5/0002; G16H 70/60; G16H 40/20; G06Q 50/26; G06Q 10/10; G16H 50/80; G16H 70/40; G16H 70/20

(57) ABSTRACT

Methods, systems, and computer-readable media are disclosed herein for Electronic Medical Record (EMR) integrated electronic case reporting. A trigger code is received from a platform. The trigger code corresponds to an infectious disease or a public health concern, such as receiving a vaccination. A patient code is also received. The patient code corresponds to information from a patient encounter. The trigger code is compared to the patient code and it is determined whether a match exists. Upon determining that the match exists, a time logic is applied for generating an electronic Initial Case Report (eICR). The time logic corresponds to the information from the patient encounter. The eICR comprising EMR data is generated.

### BACKGROUND

An electronic Initial Case Report (eICR) is a consensus-based Health Level Seven (HL7) international standard developed for electronic case reporting (eCR). An eICR is transmitted to the Association of Public Health Laboratories (APHL) Informatics Messaging Services (AIMS) platform. If the latest version of the Electronic Reporting and Surveillance Distribution (eRSD) within an Electronic Health Record (EHR) is not implemented and used, resulting reports of public health interests may go undetected or may not even be sent and received. Additionally, current reporting is not always timely and is not always accurate. For example, manual reports become problematic and challenging to maintain when more conditions are added. Accordingly, it is desirable to generate reports implementing the latest version of the eRSD and that are timely, more accurate, and maintainable for various conditions.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present disclosure is defined by the claims as supported by the Specification, including the Detailed Description.

Aspects of the present disclosure relate to methods, systems, and computer-readable media for Electronic Medical Record (EMR) integrated electronic case reporting. A trigger code is received from a platform. A patient code corresponding to EMR data of a patient comprising encounter information is received. The trigger code is compared to the patient code. Based on the comparison, it is determined that a match exists. Upon determining that the match exists, it is determined that the patient has an infectious disease or has received a particular treatment. In response to determining that the patient has the infectious disease, an eICR is generated.

### BRIEF DESCRIPTION OF THE DRAWINGS

Illustrative aspects of the present invention are described in detail below with reference to the attached drawing figures, and wherein:

FIG. 1 illustrates a computing environment, in accordance with aspects;

FIG. 2 illustrates a system for generating an eICR document, in accordance with aspects;

FIG. 3 illustrates three example phases for generating an eICR document, in accordance with aspects;

FIG. 4 illustrates example options of an example eCR application, in accordance with aspects;

FIG. 5 illustrates an example table of contents for a generated report, in accordance with aspects;

FIG. 6 illustrates example sections and headers corresponding to the generated report, in accordance with aspects;

FIG. 7 illustrates an example initial public health case report, in accordance with aspects; and

FIG. 8 illustrates an example flowchart, in accordance with aspects.

### DETAILED DESCRIPTION

The subject matter of the present invention is being described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Terms should not be interpreted as implying any particular order among or between various steps herein disclosed unless and except when the order of individual steps is explicitly described. As such, although the terms “step” and/or “block” can be used herein to connote different elements of system and/or methods, the terms should not be interpreted as implying any particular order and/or dependencies among or between various components and/or steps herein disclosed unless and except when the order of individual steps is explicitly described. The present disclosure will now be described more fully herein with reference to the accompanying drawings, which may not be drawn to scale and which are not to be construed as limiting. Indeed, the present invention can be embodied in many different forms and should not be construed as limited to the aspects set forth herein. Further, it will be apparent from this Detailed Description that the technological solutions disclosed herein are only a portion of those provided by the present invention. As such, the technological problems, solutions, advances, and improvements expressly referenced and explained herein should not be construed in a way that would limit the benefits, improvements, and/or practical application of the discussed aspects of the present invention.

Accordingly, a system, method, or medium for EMR integrated electronic case reporting provides numerous advancements over prior systems, methods, and media. As one example, present embodiments provide for updating logic for eICR generation and compliance at a faster speed than prior systems. For example, embodiments provide for communication with various types of databases; whereas in prior systems, communication with only a certain type of database was required. As another example, embodiments provide for receiving and updating trigger codes from platforms (e.g., AIMS) via various application programming interface (API) calls (e.g., Fast Healthcare Interoperability Resources (FHIR) API); whereas prior systems and methods were not FHIR API compatible or only used one type of API call.

Additionally, prior reporting methods and systems are not always timely and are not always accurate. For example, prior systems have failed to provide additional checks for testing results, diagnosis results, vaccination confirmations, and additional verifications based on timers set for checking after an initial check. Failure to detect and determine results and confirmations after an initial check results in delays to generating and transmitting an eICR. Further, prior systems have not used FHIR for validating results and confirmations. Furthermore, prior systems have not used FHIR for generating an eICR.

Generating these reports reduces burdens on providers, minimizes follow-up investigation phone calls and paperwork, and provides information to healthcare providers about an identified reportable condition. For example, these reports provide information including notices of disease outbreaks, infection control information, and next-step testing and patient management. Providing these reports can help reduce disease outbreaks and assist a community in controlling a disease outbreak. Further, submitting data to public health agencies is burdensome and disruptive to clinician workflows. As such, eCR improves timeliness, collection, completeness of disease and condition reporting, and reduces burdens to clinician workflows. Generating an eICR effectively and timely results in earlier implementation of an intervention and reduction of further spread and exposure. Furthermore, an eCR that includes EMR implemented data may include information that is not usually found in clinical electronic documents.

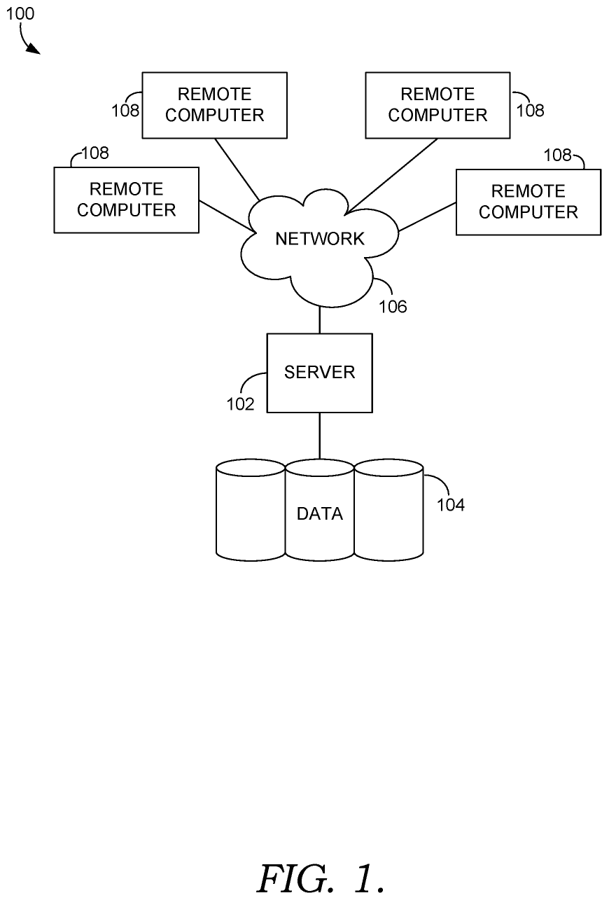

Turning now to FIG. 1, an example computing environment 100 that is suitable for use in implementing aspects of the present invention is depicted. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. Generally, in aspects, the computing environment 100 is a medical-information computing-system environment. However, this is just one example and the computing environment 100 can be operational with other types, other kinds, or other-purpose computing system environments or configurations. Examples of computing systems, environments, and/or configurations that might be suitable for use with the present invention include personal computers, server computers, hand-held or laptop devices, wearable devices, multiprocessor systems, microprocessor-based systems, set top boxes, programmable consumer electronics, network PCs, minicomputers, mainframe computers, distributed computing environments that include any of the above-mentioned systems or devices, and the like.

In aspects, the computing environment 100 can be described in the general context of computer instructions, such as program modules, applications, and/or extensions, being read and executed by a computing device. Examples of computer instructions can include routines, programs, objects, components, and/or data structures that perform particular tasks or implement particular abstract data types. The aspects discussed herein can be practiced in centralized and/or distributed computing environments, i.e., where computer tasks are performed utilizing remote processing devices that are linked through a communications network, whether hardwired, wireless, or a combination thereof. In a distributed configuration, computer instructions might be stored or located in association with one or more local and/or remote computer storage media (e.g., memory storage devices). Accordingly, different portions of computer instructions for implementing the computer tool in the computing environment 100 may be executed and run on different devices, whether local, remote, stationary, and/or mobile.

With continued reference to FIG. 1, the computing environment 100 comprises one or more computing devices in the form of server(s) 102, shown in the example form of a server. Although illustrated as one component in FIG. 1, the present invention can utilize a plurality of local servers and/or remote servers in the computing environment 100. Example components of the server(s) 102 comprise a processing unit, internal system memory, and a suitable system bus for coupling various components, including electronic storage, memory, and the like, such as a data store, a database, and/or a database cluster. Example components of the server(s) 102 include a processing unit, internal system memory, and a suitable system bus for coupling various components, including a data store 104, with the server(s) 102. An example system bus might be any of several types of bus structures, including a memory bus or memory controller, a peripheral bus, and a local bus, using any of a variety of bus architectures. Example architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The server(s) 102 typically includes therein, or has access to, a variety of non-transitory computer-readable media. Computer-readable media can be any available media that might be accessed by server(s) 102, and includes volatile, nonvolatile, removable, and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared and other wireless media. Combinations of any of the above should also be included within the scope of computer-readable media.

Server(s) 102, in some embodiments, represent a stand-alone computer or computing system, such as a mainframe, blade server, and the like. Alternatively, in some embodiments, the server(s) 102 represent a set of distributed computers, such as multiple cloud computing nodes where data is provisioned or exchanged between the cloud computing nodes. The server(s) 102 might operate in a network 106 using logical connections to one or more remote computers 108. In some aspects, the one or more remote computers 108 can be located at a variety of locations, such as medical facilities, research environments, and/or clinical laboratories (e.g., molecular diagnostic laboratories), as well as hospitals, other inpatient settings (e.g., surgical centers), veterinary environments, ambulatory settings, medical billing offices, financial offices, hospital administration settings, home healthcare environments, and/or clinicians' offices). As used herein, “clinicians,” “medical professionals,” or “healthcare providers” can include: physicians; specialists such as surgeons, radiologists, cardiologists, and oncologists; emergency medical technicians; physicians' assistants; nurse practitioners; health coaches; nurses; nurses' aides; pharmacists; dieticians; microbiologists; laboratory experts; laboratory technologists; genetic counselors; researchers; veterinarians; students; and the like.

Computer network(s) 106 comprise a local area network (LANs) and/or a wide area network (WAN). Such networking environments are commonplace in offices, enterprise-wide computer networks, intranets, and the Internet. When utilized in a WAN networking environment, the server(s) 102 might comprise a modem or other means for establishing communications over the WAN, such as the Internet. In a networking environment, program modules or portions thereof might be stored in association with the server(s) 102, the data store 104, or any of the remote computers 108. For example, various application programs may reside on the memory associated with any one or more of the remote computers 108. It will be appreciated by those of ordinary skill in the art that the network connections shown are examples and other means of establishing a communications link between the computers (e.g., server(s) 102 and remote computers 108) might be utilized.

The network 106 can include an entity-wide network, campus-wide network, an office-wide network, an enterprise-wide networks, and the Internet. In the network 106, applications, extensions, program modules or portions thereof might be stored in association with the server(s) 102, the data store 104, and any of the one or more remote computers 108. For example, various application programs can reside on the memory associated with any one or more of the remote computers 108. In the computing environment 100, which is illustrated as being a distributed configuration of the network 106, the components and devices can communicate with one another and can be linked to each other using a network 106. It will be appreciated by those of ordinary skill in the art that the network connections shown are example, and other means of establishing a communications link between the computers (e.g., server(s) 102 and remote computers 108) might be utilized.

In operation, an organization might enter commands and information, for example, directly in peer-to-peer or near-field communication, or through the network 106 using telecommunications or Wi-Fi. Other input devices comprise microphones, satellite dishes, scanners, or the like. Commands and information might also be sent directly from a remote healthcare device. In addition to a screen, monitor, or touchscreen component, remote computers 108 might comprise other peripheral output devices, such as speakers and printers. Further, in aspects where the network 106 is distributed in configuration, the one or more remote computers 108 may be located at one or more different geographic locations (e.g., located across various locations such as buildings in a campus, medical and research facilities at a medical complex, offices or “branches” of a banking/credit entity, or can be mobile devices that are wearable or carried by personnel, or attached to vehicles or trackable items in a warehouse, for example).

Turning to the data store 104, the data store 104 may be implemented using multiple data stores that are communicatively coupled to one another, independent of the geographic or physical location of a memory device. The data store 104 may also be implemented using a single data store component or may be in the cloud. The data store 104 can, for example, store data in the form of artifacts, server lists, properties associated with servers, environments, properties associated with environments, computer instructions encoded in multiple different computer programming languages, deployment scripts, applications, properties associated with applications, release packages, version information for release packages, build levels associated with applications, identifiers for applications, identifiers for release packages, users, roles associated with users, permissions associated with roles, workflows and steps in the workflows, clients, servers associated with clients, attributes associated with properties, audit information, and/or audit trails for workflows. The data store 104 can, for example, also store data in the form of electronic records, such as electronic medical records of patients, patient-specific documents and historical records, transaction records, billing records, task and workflow records, chronological event records, and the like. Generally, the data store 104 includes physical memory that is configured to store information encoded in data. For example, the data store 104 can provide storage for computer-readable instructions, computer-executable instructions, data structures, data arrays, computer programs, applications, and other data that supports the functions and actions to be undertaken using the computing environment 100 and components shown in the example of FIG. 1.

As shown in the example of FIG. 1, when the computing environment 100 operates with distributed components that are communicatively coupled via the network 106, computer instructions, applications, extensions, and/or program modules can be located in local and/or remote computer storage media (e.g., memory storage devices). Aspects of the present invention can be described in the context of computer-executable instructions, such as program modules, being executed by a computing device. Program modules can include, but are not limited to, routines, programs, objects, components, and data structures that perform particular tasks or implement particular abstract data types. Although internal components of the devices in FIG. 1 are not illustrated, those of ordinary skill in the art will appreciate that internal components and their interconnection are present in the devices of FIG. 1. Accordingly, additional details concerning the internal construction device are not further disclosed herein. Although many other internal components of the server(s) 102 and the remote computers 108 are not shown, such components and their interconnection are known. Accordingly, additional details concerning the internal construction of the server(s) 102 and the remote computers 108 are not further disclosed herein.

Additionally, it will be understood by those of ordinary skill in the art that the computing environment 100 is just one example of a suitable computing environment and is not intended to limit the scope of use or functionality of the present invention. Similarly, the computing environment 100 should not be interpreted as imputing any dependency and/or any requirements with regard to each component and combination(s) of components illustrated in FIG. 1. It will be appreciated by those having ordinary skill in the art that the connections illustrated in FIG. 1 are also examples as other methods, hardware, software, and devices for establishing a communications link between the components, devices, systems, and entities, as shown in FIG. 1, can be utilized in implementation of the present invention. Although the connections are depicted using one or more solid lines, it will be understood by those having ordinary skill in the art that the example connections of FIG. 1 can be hardwired or wireless, and can use intermediary components that have been omitted or not included in FIG. 1 for simplicity. As such, the absence of components from FIG. 1 should be not be interpreted as limiting the present invention to exclude additional components and combination(s) of components. Moreover, though devices and components are represented in FIG. 1 as singular devices and components, it will be appreciated that some aspects can include a plurality of the devices and components such that FIG. 1 should not be considered as limiting the number of a device or component.

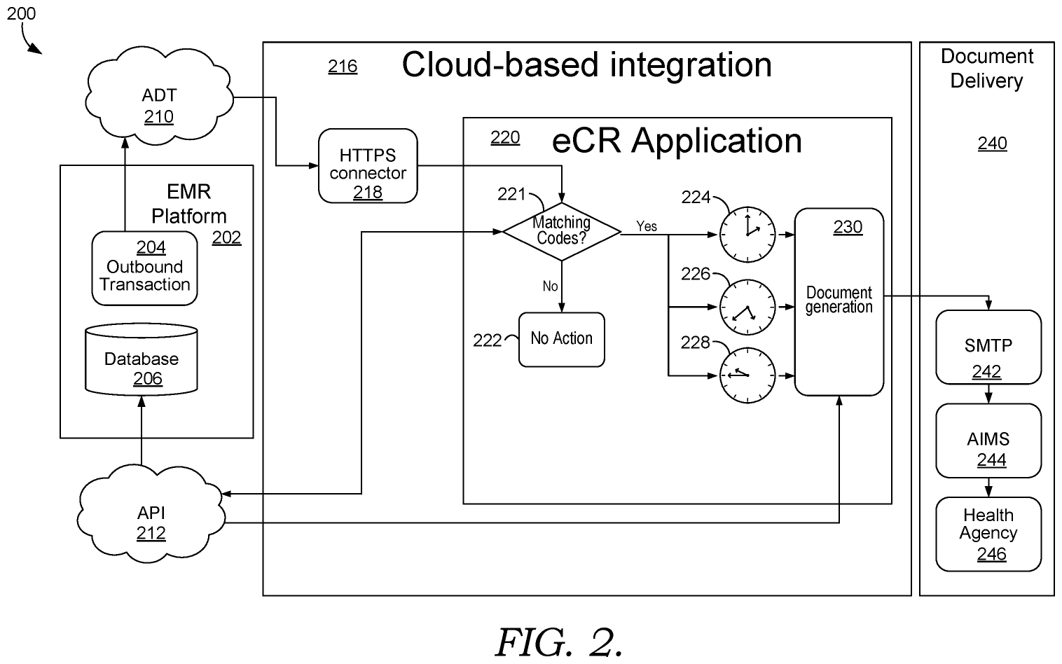

Turning now to FIG. 2, example system 200 comprises a system for generating an eICR document. Example system 200 has an EMR platform 202 that comprises an outbound transaction 204 and a database 206; an admit transfer discharge (ADT) communicator 210; an API platform 212; a cloud-based integration 216 platform; an HTTPS connector 218; an eCR application 220 comprising determiner 221, no action 222, timer 224, timer 226, timer 228, and document generation 230; and document delivery 240 comprising simple mail transfer protocol (SMTP) 242, AIMS 244, and health agency 246. Beginning with EMR platform 202, the EMR platform 202 may comprise a cloud-based library for medical information from small, medium, and large medical practices. Additionally, the EMR platform 202 may contain data from behavioral health facilities or independent behavioral health providers. For example, the EMR platform 202 may be a Java and cloud-based automated library (such as Cerner Millennium). In embodiments, the EMR platform 202 may service community hospitals, academic medical centers, ambulatory health services, multi-specialty groups, independent practices, and rehabilitation centers. In some embodiments, the EMR platform 202 may comprise artificial intelligence technology that learns as text is received or as dictations are received. Further, the EMR platform 202 may safeguard private medical data and administer information to partnering practices in an efficient, safe, and legal manner. Furthermore, the information may be distributed across multiple physical locations.

In addition, the EMR platform 202 may communicate with a plurality of EMR systems from various entities, hospitals, and departments. Although one database 206 is depicted, the EMR platform 202 may have multiple databases, wherein each database is for a different medical facility or department. In embodiments, the EMR platform 202 may receive or retrieve EMR data for a patient from an EMR system of a primary care provider and an EMR system of an emergency room, wherein the EMR systems are physically located in different states. Additionally, EMR platform 202 may receive or retrieve the EMR data in various formats. The various formats may include image formats, such as radiograph, computed tomography (CT), magnetic resonance imaging (MRI), Ultrasound (US), mammogram, positron emission tomography scan (PET), and nuclear scan (NM) images; montages of medical images; medical reports; voice clips, notes; and medical reports, for example. Further, EMR data may be received in other various formats, such as PDF, JPEG, GIF, PNG, DOC, XLS, PPT, MP3, WAV, HTML, XML, and various other formats. The EMR platform 202 may standardize the data received in various formats into a standard format for analysis and transmission.

As discussed above, the EMR platform 202 may communicate with the plurality of EMR systems by receiving EMR data from the plurality of EMR systems of different sources and transmitting the EMR data to the plurality of EMR systems. In some embodiments, data relating to the patient's current condition and/or patient demographics may be received directly from a user, such as the patient or a care provider, inputting such information into a user device. Some of the current patient data, such as patient variable values, may be received from one or more sensors or monitoring devices or directly from a laboratory running the laboratory procedures. Additionally, historical patient information may be received from the patient's EMR and/or from insurance claims data for the patient. For example, EMR data from in-home care services, hospitals, or any healthcare facility may be received. In an alternative embodiment, the patient's history may be received directly from the patient, such as during registration when admitted to a care facility for the current encounter or starting the current care services (such as with in-home care services). Upon receipt of the EMR data, the EMR platform 202 may transmit the EMR data and additional information to a health agency, such as a county health department, a district health authority, the Center for Disease Control and Prevention (CDC), World Health Organization (WHO), and/or a state department of health, for example.

In embodiments, the EMR data (sometimes referred to as electronic health records (EHR) data), may comprise electronic clinical documents such as images, clinical notes, orders, record systems (which store real-time or near real-time patient or user) information), summaries, reports, analyses, information received from clinical applications and medical devices (e.g., wearable, bedside, or in-home patient monitors), and/or other types of electronic medical documentation relevant to a particular patient's condition and/or treatment. The electronic clinical documents may contain various types of information relevant to the condition and/or treatment of a particular patient and can include information relating to, for example, patient identification information, images, alert history, culture results, patient-entered information, physical examinations, vital signs, past medical histories, surgical histories, family histories, histories of present illnesses, current and past medications, allergies, symptoms, past orders, completed orders, pending orders, tasks, lab results, other test results, patient encounters and/or visits, immunizations, physician comments, nurse comments, other caretaker comments, clinician assignments, and a host of other relevant clinical information. Further, in some embodiments, the EMR data comprising patient data may include patient demographic data, such as age, sex, race, nationality, socioeconomic status, marital status, and employment status and history. This data may further include the patient's insurance information, such as the insurance provider and the type of plan. Additional patient data may include previous and current home and work addresses.

Other types of EMR data comprising patient data may include current patient data and historical patient data. In some embodiments, current patient data includes data relating to the patient's labs, vitals, diagnoses, medications from a current encounter (e.g., a current admission to a healthcare facility, a current visit to an outpatient facility, or a current period of receiving home healthcare services). The current patient data may include a diagnosis and/or treatment (including medications administered or ordered and procedures performed or ordered). During the current encounter, the patient may be diagnosed or treated with a condition such as asthma, cancer, or heart disease, for example. Current patient data may further include lab results (e.g., physiological data), including vital sign data, from the current encounter. Historical patient data may include information about the patient's past encounters at the current healthcare facility or other healthcare facilities, past encounters at a post-acute care facility, etc. In some embodiments, historical patient data includes previous diagnoses, medications, and lab results. The content and volume of such information in an EMR system are not intended to limit the scope of the present disclosure in any way.

The EMR platform 202 may transmit information via an outbound transaction 204 to the ADT 210. The messages that the ADT 210 transfers (e.g., Hl7 ADT messages) may include patient demographic and visit information synchronized across healthcare systems. For example, some of the patient demographic and visit information may comprise admissions, cancellations of admissions, merged patient data, transfer information, pre-admission information, outpatient changed to an inpatient, inpatient changed to an outpatient, bed status, linked patient data, patient identifier lists, alternate patient IDs, visit numbers, etc. Additionally, the ADT 210 transfers trigger event information, including trigger codes (e.g., a unique five-digit numerical code assigned to each disease or condition). Messages the ADT 210 transfers may also include a Patient Identification segment (e.g., patient name and contact information), a Patient Visit segment (e.g., attending physician information and assigned patient location information), and an Insurance segment (e.g., primary and secondary insurance information including a relevant address). The ADT transmitted message information determine where the ADT message is sent. For example, ADT messages comprising particular information relating to a target condition or disease will be transmitted more quickly to the cloud-based integration platform 216 than ADT messages that do not comprise that information.

Upon transmission to the eCR application 220 of the cloud-based integration platform 216 (e.g., an Integration Platform as a Service (iPaaS)), the ADT message may be transmitted through an HTTPS connector 218 (e.g., an iPaaS HTTPS connector). For example, the HTTPS connector may be running on a Kubernetes cluster. Additionally, the eCR application 220 is in communication with API 212 and receives standard trigger codes from a healthcare related platform (e.g. from the CDC) via an API call. For example, the eCR application 220 communicates with Cerner Ignite APIs for Millennium via HL7 FHIR API calling. The standard trigger codes from the platform may be based on parameters and value sets in XML or JSON form. These trigger codes may be initially transmitted through an Electronic Reporting and Surveillance Distribution (eRSD). The eRSD may periodically update trigger codes at particular effective start dates (dates that the code or set of codes need to be implemented and in use by reporters). Sometimes the updates occur upon scheduled releases, which have effective dates that begin about four to six months from the release date. The eCR application 220 may use API calls to check for these updates. The eCR application 220 may transmit an API call upon a threshold period of time for detecting updates. In some embodiments, the eCR application 220 may implement the updated trigger codes automatically upon detection of the update.

Further, the standard trigger codes may correspond to a National Notifiable Diseases Surveillance System Event Code List. The standard trigger codes may comprise unique five-digit numerical codes that correspond to the following: acanthamoeba disease, acute flaccid myelitis, anthrax, babesiosis, balamuthia mandrillaris disease, blastomycosis, botulism, brucellosis, cache valley virus disease, California encephalitis virus disease, California serogroup virus diseases, campylobacteriosis, candida auris, CP-CRE, carbon monoxide poisoning, chancroid, chikungunya virus diseases, Chlamydia trachomatis infection, coccidioidomycosis, Colorado tick fever virus disease, COVID-19, Crimean-Congo hemorrhagic fever, cryptosporidiosis, dengue, diphtheria, eastern equine encephalitis virus disease, Ebola hemorrhagic fever, ehrlichia chaffeensis, ehrlichia ewingii, enterotoxigenic E. coli, flavivirus disease, giardiasis, gonorrhea, guanarito hemorrhagic fever, Haemophilus influenzae, Hansen's disease, Hantavirus infection, Hantavirus pulmonary syndrome, hemolytic uremic syndrome, Hepatitis A, Hepatitis B, Hepatitis C, Hepatitis E, histoplasmosis, influenza-associated pediatric mortality, invasive pneumococcal disease, Jamestown Canyon virus disease, Lassa fever, leptospirosis, listeriosis, rubeola, smallpox, syphilis, tuberculosis, West Nile virus disease, Zika virus disease, etc. Additionally, the standard trigger codes may comprise a unique code that corresponds to the following vaccinations, for example: AVA (for anthrax), Vaxchora (for cholera), DTaP (for diphtheria), Td (for diphtheria), DT (for diphtheria), Tdap (for diphtheria), DTaP-IPV (for diphtheria), DTaP-HepB-IPV (for diphtheria), HepA-HepB, HepA, HepB, HPV9, seasonal influenza vaccines, MMR (for measles, mumps), MMRV (for measles, mumps), COVID-19 vaccines, Rabies vaccines, RZV (for shingles), Vaccinia (for smallpox), typhoid fever vaccinations, etc.

At determiner 221, the eCR application 220 determines whether trigger codes received from the platform (e.g., from AIMS) match patient codes (e.g., patient codes from an EMR, from multiple EMRs, from sensors in communication with one of the multiple EMRs, from sensors in communication with the eCR application 220, from an application in communication with the eCR application 220). For example, the eCR application 220 may use deterministic record linkage to weigh for exact agreements of the five-digit numerical codes. In some embodiments, the eCR application 220 may use a probabilistic weight determination based on statistical models using probabilities that the codes match or probabilities that the codes do not match. In some embodiments, matching may be determined using a frequency of linkage variables values within a dataset. Analyzing the frequency of linkage variables may involve a string comparison, a weight determination, and/or scalability. In some embodiments, duplicate records are be purged. In some embodiments, only pairs of codes, values, or variables that agree on one or more blocking parameters are compared for matches, which reduces computation burden and hastens analysis. In some embodiments, fuzzing matching may be used (e.g. for codes that comprise phonetic similarities, abbreviations, or inadvertent entries that are incorrect).

In some embodiments, patient codes correspond to patient encounters. For example, a patient code may be generated upon admission to a hospital for symptoms that are similar or identical to a public health reportable condition (e.g., a disease, a virus, a bacterium, etc.). In some embodiments, a patient code may be generated upon a patient taking a test for the public health reportable condition. In some embodiments, a patient code may be generated upon a patient scheduling an appointment for receiving a vaccination. In some embodiments, a patient code may be generated upon a patient's primary care doctor scheduling an appointment with a specialist for the public health reportable condition.

If the eCR application 220 determines that a match does not exist, no action 222 is taken. If the eCR application 220 determines that a match does exist, a time logic for generating a report for the public health reportable condition is applied. In some embodiments, the time logic corresponds to a status of a particular test administered to the patient that corresponds to the public health condition. For example, if the public health condition is COVID-19, a first time logic 224 may be two hours, the second time logic 226 may be 12 hours, and the third time logic may be 24 hours. Continuing the example, the eCR application 220 may continuously check for a positive test result of COVID-19 every two hours for the first day, then check for the positive test result every twelve hours for the next three days, and then check every twenty-four hours for the subsequent two days. In some embodiments, the time logic corresponds to a status of a particular vaccination administered to the patient that corresponds to the public health condition. For example, the eCR application 220 may continuously check for whether the vaccination was administered every two hours for the first day, then check for a second administration of the vaccination every five hours for a day that the second vaccination was administered, and then check every twenty-four hours for a verification or a validation of a completed vaccination. The validation may be through the FHIR. For example, the validation may comprise a cardinality check that all properties are correct (e.g. a JSON schema generated for a profile that tests cardinality) or a structure check corresponding to the content of a resource providing a result of a test.

Subsequently, the eCR application 220 generates a document 230 (e.g. HL7 CDA eICR) for transmission. The first time logic 224, the second time logic 226, and the third time logic 228 may still continue running to provide further updates for subsequent document generation. FHIR, an API gateway, an ADT, and a database may be used by the eCR application 220 in generation of the document 230. In an embodiment, generation of the document 230 may comprise the following: <ClinicalDocument xmlns:sdtc=“urn:h17-org:sdtc” xmlns:cda=“urn:h17-org:v3” xmlns=“urn:h17-org:v3” xmins:xsi=“http://www.w3.org/2001/XMLSchema-instance”> <realmCode code=“US7> <typeld extension=“POCD_HD000040” root=“2.16.840.1.113883.1.3”/> <templateId root=”2.16.840.1.113883.10.20.22.1.17> <templateId extension=“2015-08-01” root=“2.16.840.1.113883.10.20.22.1.1”/> <templateId extension=“2016-12-01” root=“2.16.840.1.113883.10.20.15.2”/> <id root=“42003d69-4553-4044-a61b-afd824da4056”/> <code code=“55751-2” displayName=“Initial Public Health Case Report” codeSystemName=“LOINC” codeSystem=“2.16.840.1.113883.6.1”/>. In some embodiments, generation of the document 230 may involve source code from GitHub. The eCR application 220 allows for the EMR to be a central host for the backend. The generated document 230 may be stored locally and receipts of local storage and processing may be generated.

Document generation provides for management of cases, contact tracing, situational awareness, coordination of response measures, and connecting results from various databases. The eCR application 220 enables non-eCR enabled EMRs to rapidly implement automated document generation for transmission to a health agency. Additionally, the eCR application 220 enables document generation for routing data to appropriate public health surveillance systems without having to wait for software releases from the corresponding public health surveillance system. The eCR application 220 provides a single interface for various EMRs comprising differing formats and data (e.g. epidemiologic and lab data) for state and federal law compliance with eCR reporting by keeping definitions up to date as those definitions change during outbreaks and as the public health agency roles adjust during public health emergencies.

Upon generation of document 230, the eCR application 220 may provide for document delivery 240. For example, the document may be transmitted via SMTP 242 electronic mail transmission to AIMS 244, and subsequently to a health agency 246 (e.g. a state or a local health agency). Transmission of the reports allows for public access to population level information for making clinical and business decisions based upon the data provided. By efficiently providing reports to community and governmental agencies, the eCR application 220 provides for support to public health investigation and control efforts to manage high risk.

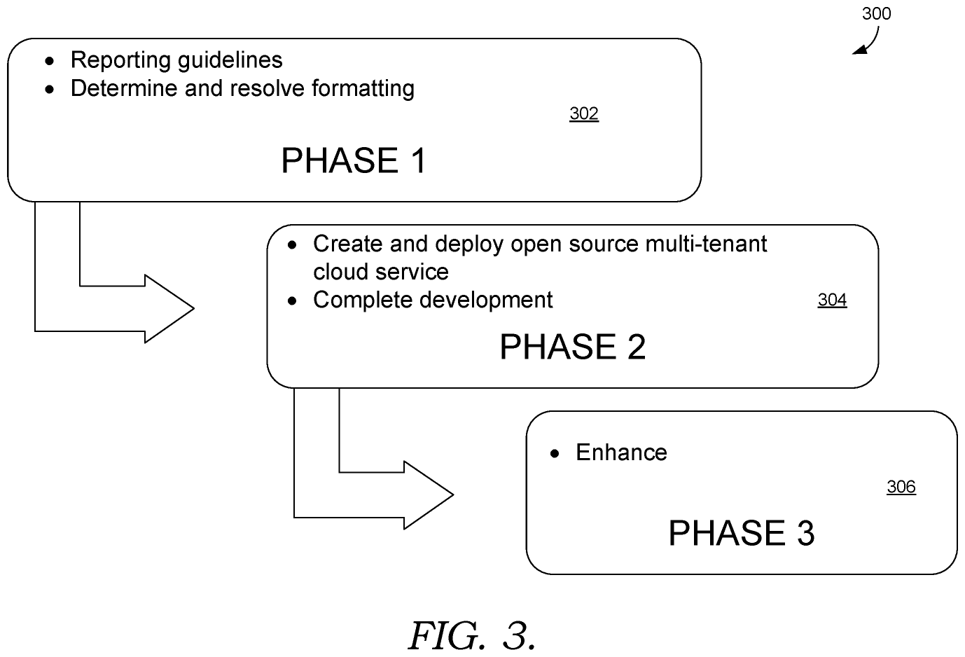

Turning now to FIG. 3, diagram 300 provides for example phase 1 at 302, example phase 2 at 304, and example phase 3 at 306 for electronic document generation. For example, at 302, phase 1 may include analyzing and implementing reporting guidelines (e.g. meeting COVID-19 CDC reporting guidelines for sending an eCR to AIMS). The in some embodiments, an EMR of a healthcare provider may already be enabled for eCR and implementation comprises enabling automation of a COVID-19 eCR. In some embodiments, the EMR is not enabled and phase 1 comprises rapid implementation for automated eCR. Implementing the reporting guidelines includes using one or more API calls to receive information from a health agency or institution for constructing an electronic case report. In some embodiments, trigger codes may be received using a first API call to a first domain and a second API call to a second domain, wherein the first domain and the second domain are different domains. Further, the trigger codes related to a particular infectious disease or vaccination may be updated based on at least two different API calls, wherein at least one of the at least two different API calls is a FHIR API call.

Phase 1 at 302 also comprises resolving formatting and updating in response to resolving the formatting. For example, different EMR systems will transmit information in various formats and the health agency or institution will transmit information in various formats. Accordingly, formatting issues between receiving trigger code information and information related to document generation and various EMR systems are determined and resolved for the proper transmission of eCR. Further, formats to eCR documents may vary from state to state, and these formatting issues are also resolved for the proper transmission of eCR.

Phase 2 at 304 comprises creating and deploying an open source multi-tenant cloud service that reads eRSD trigger codes, validates matches between eRSD trigger codes and patient codes generated during a patient encounter, generating an eICR using FHIR, sending the eICR via direct SMTP, and maintaining timers to send eICR updates. At 304, the open source multi-tenant cloud service determines an degree that existing logic is reporting documents that aligns with reporting rules, determines whether trigger codes are aligned with EMR workflow data, determines whether the document generated is accurate and complete, determines whether detection of a reportable condition was accurate and complete. In some embodiments, the open source multi-tenant cloud service determines whether EMR data comprising demographical data may be automatically linked with encounter-based data (e.g., lab results and immunization results). The open source multi-tenant cloud service continuously collects and analyzes data. In some embodiments, phase 2 is automated via a workflow component (e.g., Cerner EHR PowerChart Touch). In some embodiments, phase 2 encompasses manual clicks.

Phase 3 at 306 comprises reporting the generated document, receiving a receipt of submission, auditing, receiving updated trigger codes, and further enhancing the process. In some embodiments, health providers may submit feedback comprising testing information for further enhancing the open source multi-tenant cloud service. For example, the testing information may comprise data from FHIR sandbox that tests applications in multisource, vendor-agnostic environments. The testing information may also comprise FHIR sandbox analyses on EMR data sets transmitted to the open source multi-tenant cloud service, such as data that a health provider or patient accesses. For example, the health provider may use particular data sets for medication lists and growth charts and the patient uses data sets to become more involved in their own health care. In some embodiments, the auditing may comprise analysis of the speed at which the document was transmitted to an APHL AIMS platform.

Further, reportable conditions vary in each state and vary by territory nationally. For example, reporting laws are different in jurisdictions (e.g., what to report and when to report). Documentation requirement may also require reporting to public health agencies a residency of a patient, and this may be challenging for EMR implementers. Reporting compliance among EMR systems is not consistent and often reports do not include adequate clinical data for complete reporting. To solve these issues, the open source multi-tenant cloud service provides a single place for EMR implementers to send eCRs for routing to an appropriate jurisdiction. The open source multi-tenant cloud service automate reporting to speed up the process and to enable healthcare providers to meet legal obligations. EMR implementers of the open source multi-tenant cloud service determines when to trigger a report to be sent, determines structure and test HL7 eICR standards, receives and implements HL7 reportability response standards, and connects and tests eICR and reportability response transport and exchange.

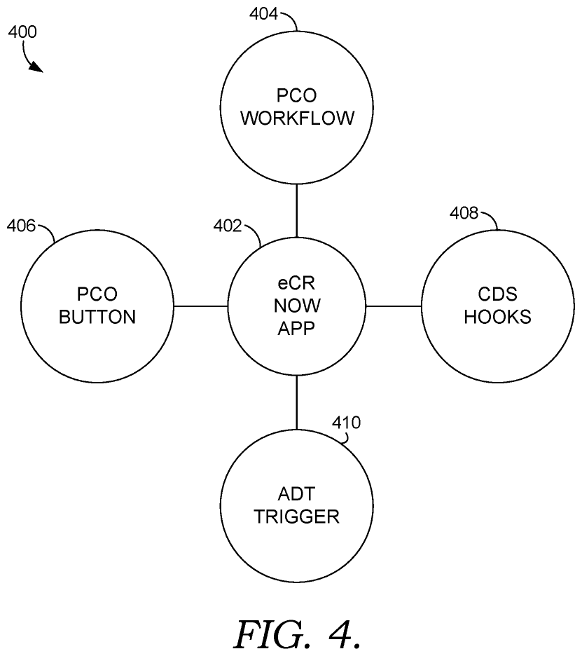

Turning to FIG. 4, environment 400 comprises eCR application options of an eCR NOW Application 402, which include Patient Centered Outcomes (PCO) workflow 404, PCO button 406, CDS Hooks 408 (an open source specification focused on user-facing remote clinical decision support with an architecturally independent specification), and ADT Trigger 410. PCO workflow 404 may determine whether trigger codes are aligned with EMR workflow and data. PCO button 406 may be used for ordering labs, radiology, scheduling, etc. CDS Hooks 408 may be used for testing services against an FHIR server for implementation of the eCR application. ADT Trigger 410 is based on reporting parameters and trigger code value sets in XML or JSON form distributed through an eRSD. EMR implementers register and subscribe to eRSD service to receive routine and emergent updates and notifications of the routine and emergent updates.

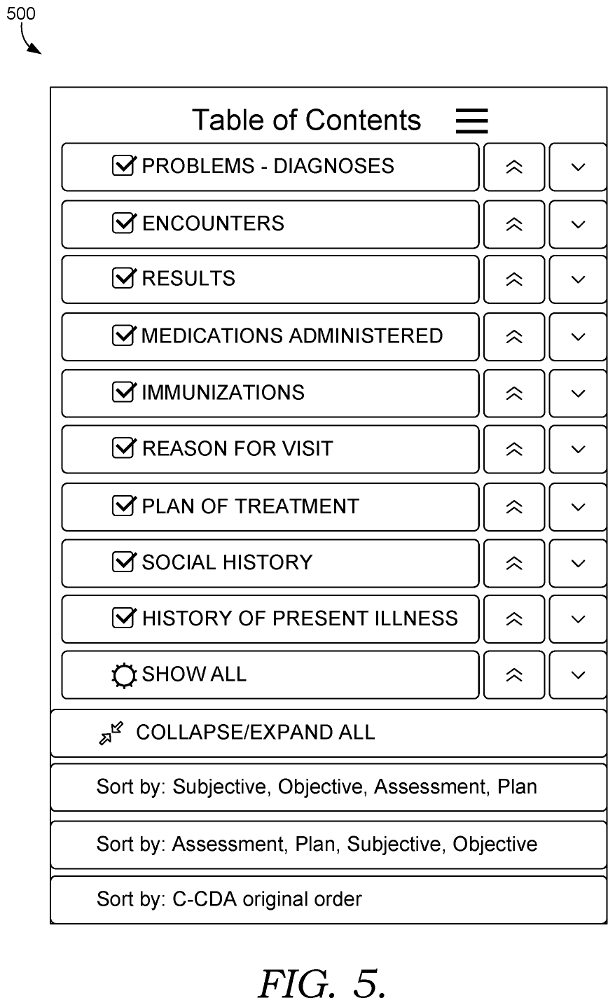

Turning now to FIG. 5, table of contents 500 may comprise EMR data, such as problems and diagnoses, patient encounters, results, medications administered, immunization provided, reasons for a visit, plan for a treatment, social history, and history of present illnesses. The table of contents 500 may be used for including EMR data into a generated electronic document. For example, the logic for generating the document may search for specific headers within specific sections of stored EMR data. Further, the table of contents 500 may also be used for organizing EMR data that was included into the generated electronic document. A domain (e.g., a Cerner Millennium domain) may be used for creating the document and structuring the document.

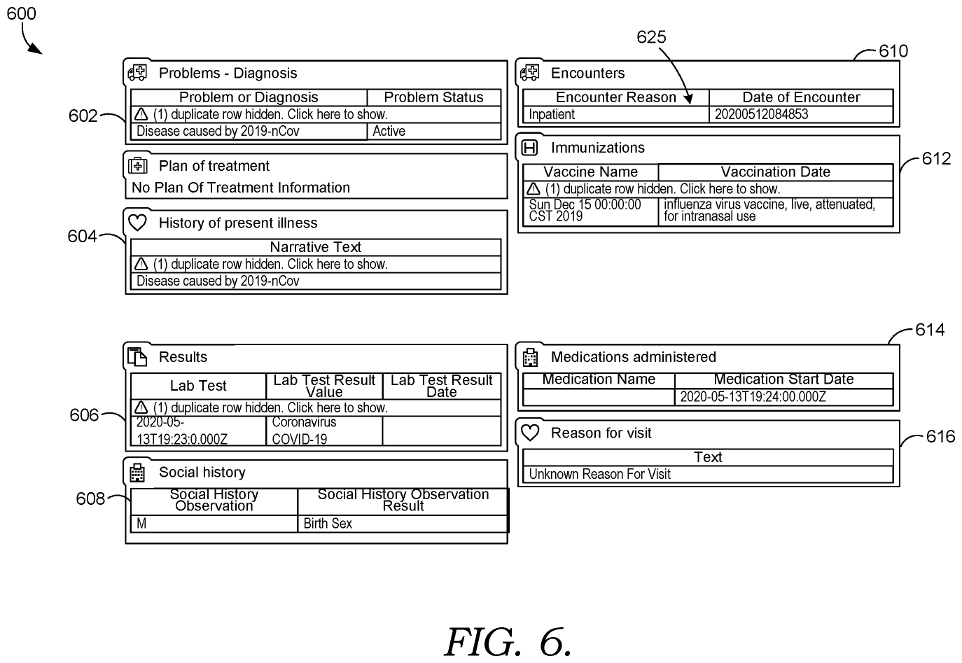

Turning now to FIG. 6, environment 600 comprises specific headers within specific sections of stored EMR data for inclusion into the generated electronic document. For example, specific headers within specific sections may include problems and diagnoses 602, history of a present illness 604, results 606, social history 608, encounters 610, immunizations 612, medications administered 614, and reasons for visits 616. Additionally, specific section with the immunizations 612 header may comprise details 625 including a date of the encounter, a reason for the encounter, and an impatient number. The sections and detailed information may be gathered from differing EMR systems for differing providers. For example, formatting of lab diagnoses may be different when collected from different domains from differing providers.

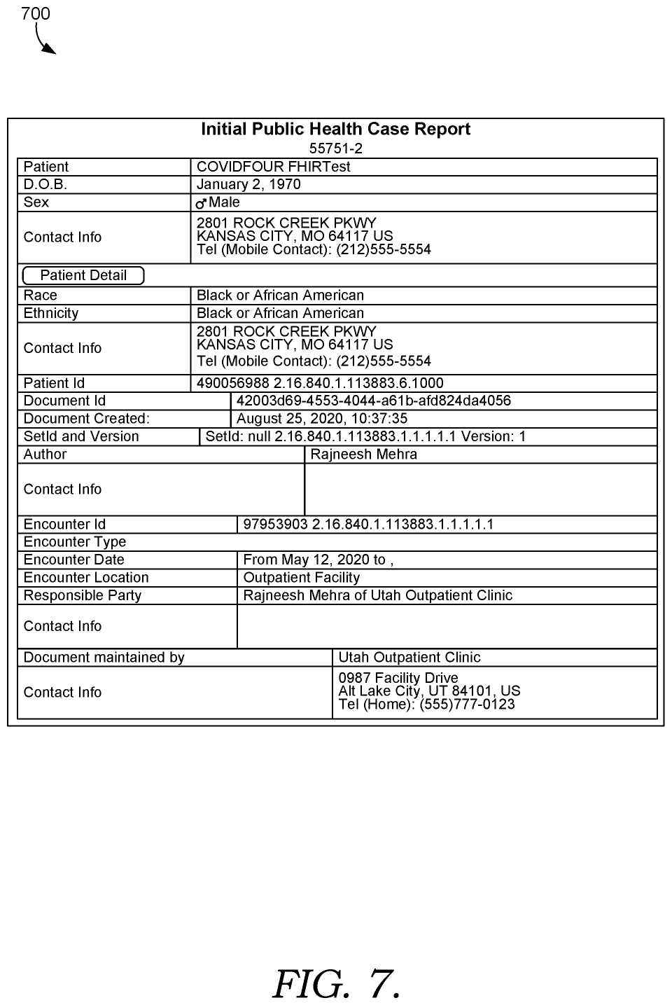

Turning now to FIG. 7, example eICR 700 is illustrated. As illustrated in example eICR 700, information in the eICR 700 may include clinical and demographic patient information, such as a residence of the patient. Example eICR 700 may be generated and sent to AIMS platform for confirmation of reportability when a laboratory order for reportable conditions, a laboratory test, a laboratory result code, or a medication matches a specific trigger code set of an eRSD. To illustrate, scenarios for generating eICR 700 include data in an EMR matching an RCTC within the eRSD. The data in the EMR may include a diagnosis that matches a value within a “diagnosis_problem” value set, a laboratory result report received by the EMR that matches a value within a “lab observation test name” value set, a laboratory order received by the EMR that matches a value within a “lab order test name” value set, or a prescribed medication received by the EMR that matches a value within a “medication” value set.

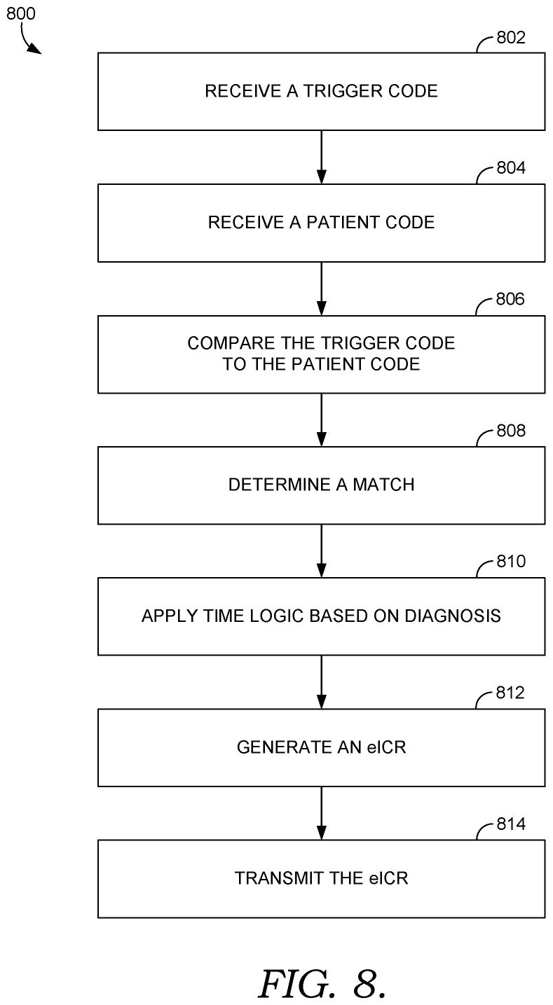

Turning now to FIG. 8, flowchart comprises step 802 for receiving a trigger code. The trigger code may be received from a platform (e.g., AIMS). Receiving the trigger code may comprise a first API call to a first domain and a second API call to a second domain, wherein the first domain and the second domain are different domains corresponding to different entities (e.g., a domain for an emergency facility and a primary care facility). The trigger code may correspond to an infectious disease to be tested during a patient encounter, wherein the infectious disease is a public health reportable condition. The infectious disease may comprise at least one of COVID-19, Chlamydia trachomatis infection, gonorrhea, pertussis, and salmonella. The determinations that the trigger code was updated by the platform may be made, and the trigger codes may then be updated based on those determinations. Determining the platform has updated the trigger code may comprise using at least two different API calls.

At 804, a patient code is received. The patient code may correspond to a patient encounter, such as a visit to a hospital, a visit to a primary care provider, a drive-through testing service, a mail-in testing service, etc. The patient code may correspond to encounter information of a patient during the patient encounter, wherein a test for the infectious disease was conducted during the patient encounter. The patient code may be received via an HTTPS connection after transmission via an ADT message. The HTTPS connection may be running on a Kubernetes cluster. Further, at 806, the trigger code is compared to the patient code. In some embodiments, multiple trigger codes are compared to multiple patient codes for the same patient. Based on the comparison or based on multiple comparisons, a match is determined at 808. For example, data in an EMR system, including a diagnosis, may match a value within a “diagnosis_problem” value set of the trigger codes.

At 810, a time logic is applied in response to determining a match exists. In some embodiments, the time logic is based on a diagnosis of the patient or a test corresponding to the infectious disease. In some embodiments, the time logic is based on various encounter information of the patient. Applying the time logic may comprise scanning for a positive result of the test after a period of time until the patient encounter is closed. Additionally, upon determining that an additional match exists, a second time logic may be applied for generating a second eICR, the second time logic based on received EMR data. The second time logic may correspond to the same test, diagnosis, or patient encounter information that the first time logic was based on. The second time logic may also correspond to a different test, a different diagnosis, or additional patient encounter information that the first time logic was based on. In some embodiments, a timer may end when a patient has been discharged. In some embodiments, a second timer may end within a certain time period after the patient has been discharged. In some embodiments, a timer may correspond to receiving a validation of a test result for the infectious disease. The validation of the test for the infectious disease may be done through FHIR.

At 812, an eICR is generated based on the time logic. The eICR may be generated after an indication that the patient has received a particular treatment, wherein the treatment is a particular vaccination. In some embodiments, the eICR is based on a determination of a positive test for the infectious disease. The eICR may comprise EMR data corresponding to one or more patients the eICR was generated for. The eICR may include demographic information, lab work information, diagnoses, observations, social history, problems, etc. One or more domains may be used for generation of the eICR. The eICR may also comprise an encounter date, an encounter location, contact information of the one or more patients, and an ethnicity of the one or more patients. The eICR may comprise a first section for immunizations and a second section for medications administered. Further, at 814, the eICR may be transmitted to a state or federal health agency. In some embodiments, the eICR may be transmitted to an educational institution. In some embodiments, the eICR may be transmitted to global health institution, such as WHO.

The present invention has now been described in relation to particular aspects, which are intended in all respects to be illustrative rather than restrictive. Thus the present invention is not limited to these aspects, but variations and modifications can be made without departing from the scope of the present invention.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A method for generating an Electronic Medical Record (EMR) integrated electronic case report, the method comprising:
receiving a trigger code from a platform, the trigger code corresponding to an infectious disease to be tested during a patient encounter, wherein the infectious disease is a public health reportable condition;
receiving a patient code corresponding to encounter information of a patient during the patient encounter, wherein a test for the infectious disease was conducted during the patient encounter;
comparing the trigger code to the patient code;
based on comparing the trigger code to the patient code, determining that a match exists;
upon determining that the match exists, applying a time logic for generating an electronic Initial Case Report (eICR), the time logic based on a status of the test; and
generating the eICR for the patient, the eICR comprising EMR data of the patient.

2. The method of claim 1, wherein the patient code was received via a hypertext transfer protocol secure (HTTPS) connection after transmission via an Admit Discharge Transfer (ADT) message.

3. The method of claim 2, wherein the HTTPS connection is running on a Kubernetes cluster.

4. The method of claim 1, wherein the infectious disease comprises one of coronavirus disease 2019 (COVID-19), Chlamydia trachomatis infection, gonorrhea, pertussis, and salmonella.

5. The method of claim 1, further comprising validating, prior to generating the eICR for the patient, the test for the infectious disease through Fast Healthcare Interoperability Resource (FHIR).

6. The method of claim 1, further comprising transmitting the eICR for the patient to a state or federal health agency.

7. The method of claim 1, wherein the status of the test comprises at least two confirmations of existence of the infectious disease, and wherein the platform is an Association of Public Health Laboratories (APHL) Informatics Messaging Services (AIMS) platform.

8. The method of claim 1, wherein the eICR comprises a first section for immunizations and a second section for medications administered.

9. The method of claim 1, wherein applying the time logic comprises scanning for a positive result of the test after a period of time until the patient encounter is closed.

10. A non-transitory computer-readable storage medium having instructions embodied thereon, the instructions being executable by one or more processors to perform a method for generating an EMR integrated electronic case report, the method comprising:
receiving a trigger code from a platform, the trigger code corresponding to an infectious disease that is a public health reportable condition;
receiving a patient code corresponding to EMR data of a patient comprising encounter information corresponding to the infectious disease;
comparing the trigger code to the patient code;
based on comparing the trigger code to the patient code, determining that a match exists;
upon determining that the match exists, applying a time logic for generating an eICR, the time logic based on received encounter information;
determining that the patient has the infectious disease based on the received encounter information; and
in response to determining that the patient has the infectious disease, generating the eICR for the patient.

11. The media of claim 10, further comprising transmitting the eICR to a health agency.

12. The media of claim 10, wherein the eICR comprises an encounter date, an encounter location, contact information of the patient, and an ethnicity of the patient.

13. The media of claim 10, further comprising:
receiving a set of trigger codes from the platform, the set of trigger codes corresponding to a second infectious disease that is a second public health reportable condition;
receiving a set of patient codes corresponding to EMR data of a second patient corresponding to the second infectious disease;
comparing the set of trigger codes to the set of patient codes;
based on comparing the set of trigger codes to the set of patient codes, determining that a second match exists;
upon determining that the second match exists, applying a second time logic for generating a second eICR, the second time logic based on received EMR data;
determining that the second patient has the second infectious disease based on the received EMR data; and
in response to determining that the second patient has the second infectious disease, generating the second eICR for the patient.

14. The media of claim 10, further comprising:
determining the platform has updated the trigger code; and
updating the trigger code based on determining the platform updated the trigger code.

15. The media of claim 14, wherein determining the platform has updated the trigger code comprises using at least two different (Application Programming Interface) API calls.

16. The media of claim 14, wherein receiving the trigger code comprises a first API call to a first domain and a second API call to a second domain, wherein the first domain and the second domain are different domains.

17. A system for generating an EMR integrated electronic case report, the system comprising:
one or more processors; and
one or more computer storage media storing computer-useable instructions that, when executed by the one or more processors, perform operations comprising:
receive a trigger code from a platform, the trigger code corresponding to a public health concern;
receive a patient code corresponding to EMR data of a patient comprising encounter information corresponding to the public health concern;
compare the trigger code to the patient code;
based on comparing the trigger code to the patient code, determine that a match exists;
upon determining that the match exists, determine whether the patient has received a treatment based on the received encounter information;
in response to a determination that the patient has received the treatment, generate an eICR for the patient; and
in response to a determination that the patient has not received the treatment, scan the EMR data for an indication that the patient has received the treatment for a period of time after the determination that the patient has not received the treatment.

18. The system of claim 17, further comprising generate an eICR after the indication that the patient has received the treatment, wherein the treatment is a particular vaccination.

19. The system of claim 17, further comprising:
determine the platform has updated the trigger code by a first API call to a first domain and a second API call to a second domain, wherein the first domain and the second domain are different domains;
update the trigger code based on determining the platform updated the trigger code; and
store an association between the trigger code that was updated and the patient code.

20. The system of claim 17, wherein the determination that the patient has received the treatment comprises a first API call to a first domain and a second API call to a second domain.
